(19) United States
(12) Patent Application Publication
(10) Pub. No.: US 2013/0233876 A1
(43) Pub. Date: Sep. 12, 2013

(54) Bandage Dispenser

(71) Applicants: Hunter Bennett Teates, Blacksburg, VA (US); David Eric Collins, Blacksburg, VA (US)

(72) Inventors: Hunter Bennett Teates, Blacksburg, VA (US); David Eric Collins, Blacksburg, VA (US)

(21) Appl. No.: 13/796,205

(22) Filed: Mar. 12, 2013

Related U.S. Application Data
(60) Provisional application No. 61/760,051, filed on Feb. 2, 2013.
(60) Provisional application No. 61/609,602, filed on Mar. 12, 2012.

(51) Int. Cl. A61F 15/00

(52) U.S. Cl. CPC A61F 15/002
USPC 221/70; 242/588.6

(57) ABSTRACT

Systems for dispensing adhesive bandages are provided for quick one-handed retrieval and application of a bandage to a wound. The dispensers and packaging keep the bandages free from contamination. A dispenser for housing rolled dispensing tape comprising a series of partially overlapped adhesive bandages, wherein each bandage is releasably adhered to the dispensing tape only at one end is specifically provided. Also provided is a hollow dispenser housing a series of adhesive bandages disposed in a stack, wherein the bandages are adhered to one another at one end of each bandage and during use are advanced sequentially through an opening in the dispenser by pulling the free end of the top bandage in the stack. The systems may be easily manufactured and made available to the consumer or medical professional.

### CROSS-REFERENCE TO RELATED APPLICATIONS

This application claims priority to and the benefit of the filing date of U.S. Provisional Application Nos. 61/609,602, filed Mar. 12, 2012, and 61/760,051, filed Feb. 2, 2013, the disclosures of each of which are hereby incorporated by reference herein in their entirety.

### BACKGROUND OF THE INVENTION

1. Field of the Invention

The present invention relates to adhesive bandages and in particular to systems for dispensing adhesive bandages. Specifically, the invention is directed to systems for dispensing adhesive bandages arranged in a series, either on a dispensing tape or in a stacked configuration.

2. Description of the Related Art

A traditional means for packaging bandages that is widely used on the market today is a box containing individual bandages which must be retrieved one at a time. This type of dispenser has not substantially changed in decades. Each bandage retrieved is sealed in a paper envelope that must be torn open and the bandage removed. Once removed, the bandage still has two lightly fixed protective coverings that must be removed before the bandage may be applied to a wound. These manipulations effectively require two hands, making it virtually impossible for a one-handed person to perform, such as a person with a hand injury in need of dressing. Such procedures are also time consuming, which severely limits the extent to which a bandage may be applied immediately after a wound to dress and protect the wound quickly, thus increasing the risk for infection. Further complicating these manipulations is that the wound to be dressed is often present on one of the fingers or hands required to retrieve the bandage, which often results in blood contaminating the packaging material or rendering the bandage useless due to it becoming wet and slippery or counteracting the adhesive on the bandage. The wrappings conventionally used to package bandages are typically paper products that are generally discarded after use, resulting in wasting of these materials.

Bandage dispensers and packaging methods that dispense a continuous series of bandages from a container in order to facilitate retrieval have been attempted. However, these dispensers and packaging methods have generally not enjoyed success on the market due to the fact that most are too complex and/or impractical for manufacture for practical use. For example, the devices disclosed in U.S. Pat. Nos. 6,213,343, 7,683,235, and 8,100,290 use gears and/or motors to dispense a series of bandages. The dispenser disclosed in U.S. Pat. No. 5,271,522, while of simpler construction, uses a tensioning device for advancing the bandages; such device is made of elastomeric material such as rubber, which can easily break. U.S. Pat. Nos. 5,358,140 and 8,052,009 describe dispensers of simpler construction, yet each provides the bandages packaged within two continuous layers of wrapping material which must be removed. The dispenser disclosed in U.S. Pat. No. 3,530,494 is operated with two hands. Additional dispensers are provided by U.S. Pat. Nos. 4,735,342, 4,993,586, 5,511,689, 5,806,714, 5,981,823, 6,225,522, 6,299,018, 6,756,519, 7,568,580, and 7,591,371, as well as U.S. Patent Application Publication Nos. 2002/0170918, 2003/0047566, and 2005/0167542, however, none of these addresses the above-mentioned issues sufficiently.

Thus, the art still has not come up with a practical solution to this problem. There remains a need in the art for bandage dispensers that are simple to use yet provide a means to quickly and intuitively extract a bandage from its container so that said bandage is immediately ready for application.

### SUMMARY OF THE INVENTION

To this end, the present invention provides systems for dispensing adhesive bandages that allow quick retrieval and application of a bandage to a wound, while simultaneously keeping the bandage free from contamination. The systems may be easily manufactured and made available to the consumer or medical professional, and use a minimal amount of materials. In embodiments, the systems allow retrieval and application of a bandage with minimal manipulation, permitting one-handed operation. The systems may be used in a variety of settings where a bandage is needed.

Generally, the systems of the invention comprise a dispensing means and a continuous series of sterile bandages packaged so that each bandage in the series may be quickly removed one at a time from the dispensing means with the thumb and forefinger of one hand for use in covering an external wound. Each bandage so dispensed is substantially free of wrapping material so that minimal manipulation is required before it may be applied. The dispensing means of the invention protects the bandages from contamination so that they are kept sterile prior to use. The systems may be used at home, on travel, at work, and in medical offices.

The dispensing means may be made of any suitable material or combination of materials, including but not limited to plastic, metal, ceramic, rubber, or cardboard, and may take a variety of shapes for dispensing the series of bandages, including but not limited to circular, oval, rectangular, or cylindrical, and may have a variety of widths to cover several different sizes of bandages, ranging from ⅛^{th} of an inch to several inches. The series of adhesive bandages may be so packaged so that individual bandages may be removed one at a time, and removal of each bandage advances the next bandage in the continuous series for later removal. Each adhesive bandage that is packaged in the invention is configured as a conventional bandage made available by various manufacturers, and includes a fixed length and width of adhesive tape that may be stuck to human skin without short or long term damage to the skin and a sterile pad of the same or near same width and shorter length that is centrally located within the bandage.

More specifically, in one aspect, the invention is a system for dispensing adhesive bandages, comprising a dispensing tape, a series of longitudinally-arrayed bandages fixed to the dispensing tape at substantially equal intervals, and a dispensing means for dispensing the dispensing tape with bandages. The dispensing tape has a planar surface and is the same width or slightly larger as each of the longitudinally-arrayed bandages, and is disposed in a roll, wherein the roll is situated within the dispensing means so that the dispensing tape may unwind from the roll. In this aspect, each bandage is consistently oriented so that it is releasably adhered to the dispensing tape at one end while the opposite end is free. Further to this aspect, each bandage substantially lacks a wrapper except for a layer disposed on the adhesive side of the bandage at its free end. In embodiments, this wrapper may enclose the free end of the bandage, including the sterile pad. Each bandage may be removed one at a time by grasping the free end with layer between the thumb and forefinger.

Specific objects of the invention thus provide a system for dispensing adhesive bandages, comprising: (a) a dispensing tape disposed in a roll; (b) a series of partially overlapping adhesive bandages fixed to the dispensing tape, wherein each of the adhesive bandages has a first end releasably adhered to the dispensing tape and a second end that is not adhered to the dispensing tape; and (c) a dispenser for housing the dispensing tape and the adhesive bandages.

Any system of the invention can provide sterile bandages with no overwrap enclosing any adhesive bandage in its entirety. Alternatively or in addition, a portion of the bandage can comprise an overwrap that encloses a portion of the bandage. In preferred embodiments, a protective cover covers one of the adhesive arms of the bandage and the sterile pad in its entirety to keep the pad sterile. For example, in embodiments, such systems can comprise a series of adhesive bandages, wherein each adhesive bandage comprises a sterile pad and has a protective cover disposed over the sterile pad. The protective cover can be releasably adhered to the adhesive bandage at only part of the first end of the bandage (e.g., on only a portion of the bottom adhesive surface of the first end), at the second end of the bandage in its entirety (e.g., covering the bottom adhesive surface of second end), and along a perimeter in its entirety surrounding the sterile pad (e.g., the adhesive portion of the bandage that surrounds the pad). In embodiments, the protective cover can have a larger perimeter than a perimeter of the adhesive bandage to which it is adhered. The protective cover can alternatively or in addition comprise one or more tabs for gripping the protective cover for removal from the adhesive bandage.

Systems of the invention can comprise dispensing tape having a planar surface and a width equal to or larger than each of the adhesive bandages. In embodiments, the dispensing tape has a leading end and an opposite end and each of the adhesive bandages is disposed lengthwise on the dispensing tape with its first end oriented toward the leading end and its second end oriented toward the opposite end of the dispensing tape. Preferred systems can comprise dispensing tape that is a strip of material made of paper or plastic.

The shape and size of the dispenser is not critical, however, to save on materials costs and manufacturing costs, having a dispenser just slightly larger than the roll or stack of bandages is preferred. In embodiments, the dispenser comprises a cylinder and the roll of dispensing tape is wrapped around the cylinder. Systems of the invention can comprise a cylinder having two ends and further comprising two disks disposed parallel to one another, one at each end of the cylinder, where the disks have a larger circumference than the ends of the cylinder. the systems can comprise a cylindrical housing operably configured for engaging the disks at their circumference and for retaining in its entirety the roll of dispensing tape disposed on the cylinder.

In another aspect, the invention is a system for dispensing adhesive bandages, comprising a series of bandages arranged in an accordion—like stack wherein the distal end of each bandage to be dispensed is releasably adhered to the proximal end of the next bandage to be dispensed, and a dispensing means for dispensing the series of bandages comprising an opening. In this aspect, each bandage substantially lacks a wrapper except for a layer disposed on the adhesive side of the bandage at its proximal end and enclosing the sterile pad. The proximal end of the bandage at the beginning of the series protrudes through the opening of the dispenser. Further to this aspect, grasping the proximal end of the bandage at the beginning of the series removes the bandage from the stack while simultaneously advancing the proximal end of the next bandage in the series through the opening.

Specific objects of the invention further provide a system for dispensing adhesive bandages, comprising: (a) a hollow dispenser with an opening; (b) a series of adhesive bandages disposed in a stack within the dispenser; (c) wherein each bandage comprises a strip of material with a top and bottom surface and adhesive disposed on the bottom surface; wherein the adhesive releasably adheres a first end of each bandage to the top surface of a second end of another bandage in the stack, and wherein the bottom surface of the second end of each bandage is not adhered to another bandage in the stack (but can be adhered to a protective cover); wherein, during use of the system, the second end of a top bandage in the stack protrudes through the opening of the dispenser and when pulled by a user the top bandage is removed from the stack and the second end of another bandage in the stack protrudes through the opening of the dispenser.

In embodiments, such systems can comprise no overwrap enclosing any adhesive bandage in its entirety. Sterility of the bandages can be provided by a protective cover, which covers the sterile pad and part of the adhesive portion of the bandage. For example, such systems can comprise bandages with a sterile pad comprising a sterile pad and having a protective cover disposed over the sterile pad, which cover is releasably adhered to the adhesive bandage at only part of the bottom surface of the first end, and at the bottom surface of the second end in its entirety, and along the bottom surface in its entirety which surrounds the sterile pad at its perimeter. Preferred are bandages with a protective cover having a larger perimeter than a perimeter of the adhesive bandage to which it is adhered. Additionally or alternatively, the protective cover can have one or more tabs for gripping the protective cover for removal from the adhesive bandage.

The dispenser can be stand alone devices or can be mounted to a substrate, which can be adhered to a wall, cabinet, table, or other surface for easy access to the bandages. In embodiments, the system can be mounted to a planar member, such as cardboard, wood, or plastic.

The dispensers can comprise a dispenser outer cover. Such an outer cover can be attached by a hinge to the planar member or attached by a hinge to the dispenser.

### BRIEF DESCRIPTION OF THE DRAWINGS

FIG. 1 is a schematic drawing depicting a top, front, right side view of an embodiment of a dispensing means for use in a system according to the invention.

FIG. 2 is a schematic drawing depicting a top view of an embodiment of a series of adhesive bandages packaged for use in a system according to the invention.

FIG. 3A is a schematic drawing depicting a top view of an embodiment of an adhesive bandage and dispensing tape for use in a system according to the invention.

FIG. 3B is a schematic drawing depicting a bottom view of an embodiment of an adhesive bandage for use in a system according to the invention.

FIG. 4A is a schematic drawing depicting a top view of another embodiment of an adhesive bandage for use in a system according to the invention.

FIG. 4B is a schematic drawing depicting a bottom view of another embodiment of an adhesive bandage for use in a system according to the invention.

FIG. 4C is a schematic drawing depicting a side view of another embodiment of an adhesive bandage for use in a system according to the invention.

FIG. 5A is a schematic drawing depicting a side view of an embodiment of a series of adhesive bandages for use in a system according to the invention.

FIG. 5B is a schematic drawing depicting a close-up side view of the embodiment shown in FIG. 5A.

FIG. 6 is a schematic drawing of a top, front, right side view of another embodiment of a dispensing means for use in a system according to the invention.

FIG. 7 is a schematic drawing depicting a top view of another embodiment of a dispensing means for use in a system according to the invention.

FIG. 8 is a schematic drawing depicting a front, top view of the embodiment shown in FIG. 7

FIG. 9 is a schematic drawing depicting a left side view of the embodiment shown in FIG. 7 and FIG. 8.

FIG. 10 is a schematic drawing depicting a right side view of the embodiment shown in FIGS. 7-9.

### DETAILED DESCRIPTION OF EMBODIMENTS OF THE INVENTION

The present invention may be carried out using a variety of means, which will be shown in the accompanying figures and which will refer to various exemplary embodiments of the invention. However, the embodiments described in the description and shown in the figures are illustrative only and are not intended to limit the scope of the invention, and changes may be made in the specific constructions described in this specification and accompanying drawings that a person of ordinary skill in the art will recognize are within the scope and spirit of the invention. Particular attention should be called to the fact that the relative size and number of bandages depicted in the following figures is merely exemplary, and the invention may accommodate a wide range of bandage sizes and numbers that may fall under its practical application.

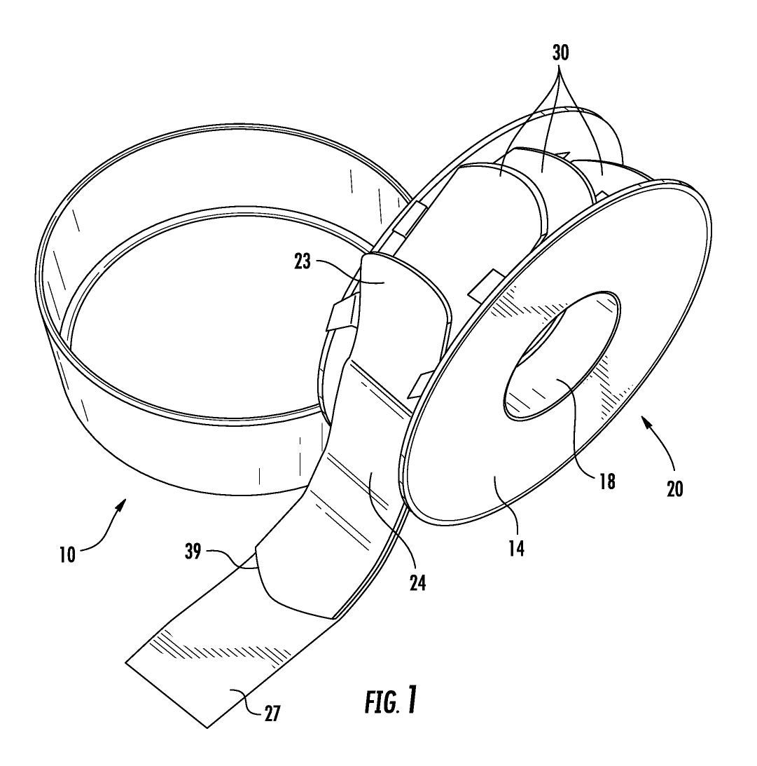

FIG. 1 shows a top, front, right side view of an embodiment of a system of the invention. A series of bandages 30 is attached to a dispensing tape 27, which together are packaged in a dispensing means 20. The dispensing means 20 comprises a cylinder 18 which the series of bandages 30 and dispensing tape 27 are rolled around. The height of the cylinder 18 (i.e. the distance between the bases) is approximately the same as the width of the dispensing tape 27. The dispensing means 20 further comprises two circular flat plates 14 arranged in parallel at both of the bases of the cylinder 18 as shown in FIG. 1, wherein the circumference of the flat plates 14 extends outward from that of the cylinder 18. The flat plates 14 may provide a space that is as wide as the height of the cylinder 18 wherein the series of bandages 30 with dispensing tape 27 may be rolled around the cylinder 18, and also provide a barrier which prevents successive layers of the roll from migrating sideways. In the embodiment shown in FIG. 1, the cylinder is open, thereby forming a circular hole within the cylinder that facilitates carrying of the dispensing means. The dispensing means 20 may be inserted into a larger cylinder 10 which is partially open. The larger cylinder 10 (outer cover or housing) may serve as a carrier for protecting dispensing means 20 and a series of bandages 30. Dispenser 20 and carrier 10 may be made of any suitable material, e.g., metal, metal alloy, plastic, or cardboard.

In preferred embodiments, and as illustrated in FIG. 1, provided is a system for dispensing adhesive bandages, comprising: (a) a dispensing tape 27 disposed as a roll; (b) a series 30 of partially overlapping adhesive bandages 24 fixed to the dispensing tape 27, wherein each of the adhesive bandages has a first end 39 releasably adhered to the dispensing tape and a second end 23 that is not adhered to the dispensing tape (but may be adhered to a protective cover); and (c) a dispenser 20 for housing the dispensing tape 27 and the series 30 of adhesive bandages 24.

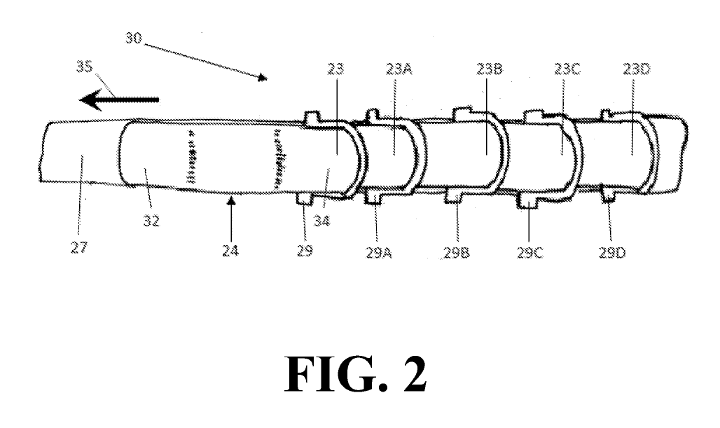

FIG. 2 shows a top view of an embodiment of the series of bandages 30 that may be used with the dispensing means 20 in a system of the invention. A series of bandages 30 at predetermined, equally-spaced intervals is attached to a dispensing tape 27. FIG. 2 shows the lead bandage 24 in the series 30 attached to a dispensing tape 27 with an adhesive at the proximal end of the dispensing tape (the direction in which the dispensing tape unwinds, shown by the large arrow 35 in the diagram).

The lead bandage 24 has a proximal end 32, which is closest to the end of the dispensing tape 27 that is the leading end for unwinding and is releasably fixed to the dispensing tape 27, and a distal end 34 which is furthest from the lead end of the dispensing tape 27 and is free of the dispensing tape 27. The lead bandage 24 comprises a finger grip area 23 and a bandage protective tab 29 at the free distal end 34. The lead bandage 24 is removed from the dispensing means 20 by grasping the finger grip area 23 and bandage protective tab 29 between the thumb and index finger and pulling the bandage free of the dispensing tape 27. The series of bandages 30 is rolled around the cylinder 18 of the dispensing means 20, so that the first bandage to be removed 24 is removed using the finger grip area 23 and bandage protective tab 29.

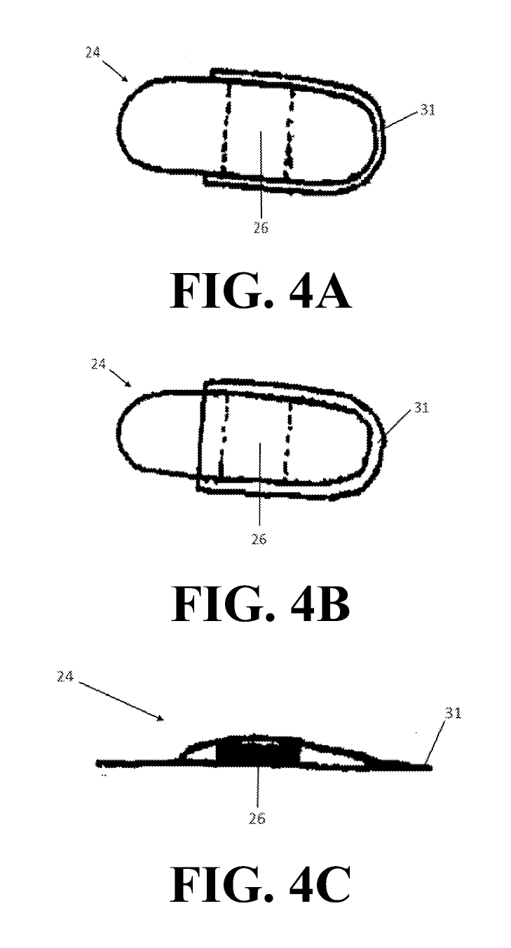

Each bandage in the series is arranged in a manner similar to that of the lead bandage 24 and in an overlapping relationship relative to lead bandage 24. Thus, after the first bandage is removed, the next bandage to be removed in the series would be removed through grasping finger grip area 23A and bandage protective tab 29A, the next bandage would be removed through grasping finger grip area 23B and bandage protective tab 29B, the next would be removed through grasping finger grip area 23C and bandage protective tab 29C, and the next would be removed through grasping finger grip area 23D and bandage protective tab 29D, and so on in the series. In this embodiment, each bandage in the series substantially overlaps the next bandage in the series, which maximizes the number of bandages that may be fixed to the dispensing tape 27. Only one end of the bandage is affixed to the dispensing tape. In embodiments, protective tab 29 can have a width that is slightly larger than the width of the finger grip area 23. Additionally, or alternatively, the protective tab 29 can have an outline that is the same shape as the portion of the bandage that it protects. The protective tab can have one or more tab-like protrusions on the side or end portion of the protective tab. As illustrated, the protective tab has square shaped side protrusions. In embodiments, the protective tab can bear instructions for the user of the device, such as “pull here,” and/or can be numbered such that the bandages are consecutively numbered from highest to lowest to serve as an indicator to the user how many bandages remain to be dispensed. The protective tab can also be configured to cover the pad to keep it sterile. In one embodiment, the adhesive strip of the bandage also surrounds the pad, such that the protective tab can adhere to all sides of the adhesive strip to completely surround and protect the pad (see FIGS. 4A-B).

This particular packaging method is suitable for a variety of widths and lengths of bandages, so long as the dispensing tape 27 is so dimensioned that it accommodates the width of the bandage that is the object of packaging. The length of dispensing tape 27 will be determined by the number of bandages that one wishes to package and the length of the particular type of bandage that is the object of packaging.

The scope of this invention may include additional variations and configurations from that shown in FIGS. 1 and 2. For example, in another embodiment, the dispensing tape with a series of bandages is reversed in orientation and the bandages are spaced so that each bandage may be removed by grasping its proximal end. Further, in other embodiments, the dispensing means may be configured similar to a conventional tape dispenser, with a flat base portion for allowing the dispenser to be positioned on a flat surface such as a desktop or countertop, a cylinder portion similar to that shown in FIG. 1, means for holding the cylinder in place, and side portions for keeping successive layers of the roll from migrating sideways. This embodiment may further provide a sharp surface for removing the end portion of the dispensing tape after bandages have been removed from it. In other embodiments, the dispensing means may include a flat portion with holes for mounting it to a wall or underneath a cabinet. These and similar constructions will be readily apparent to the skilled artisan. The dispensing means may take a variety of forms, so long as the dispensing tape 27 with series of bandages 30 may be effectively packaged in a roll. The dispensing means of the invention may range from being completely portable to fixed, depending on the varying needs of medical consumers. This provides for the dispensing means to be used in the home, workplace, on travel, as well as in clinical settings: anywhere where a quick application of a bandage may be needed.

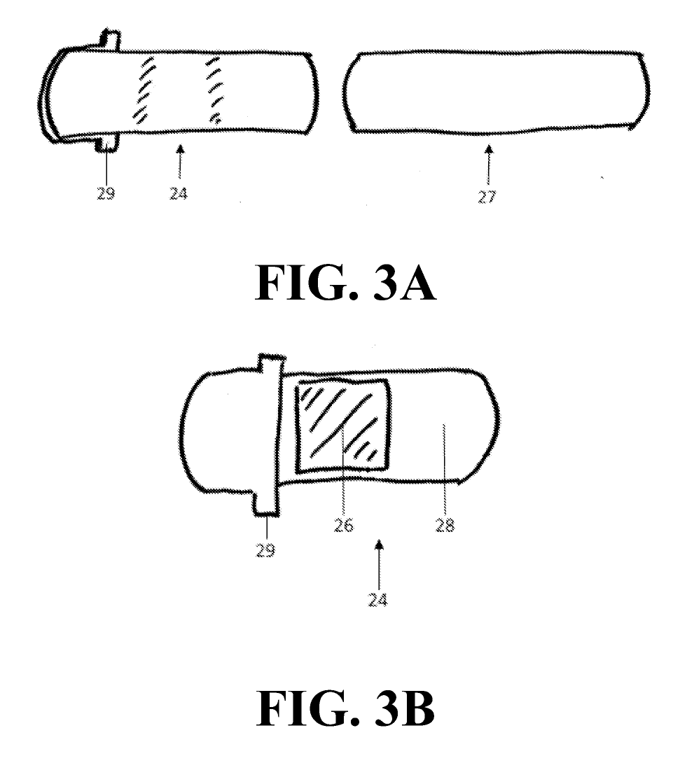

FIG. 3A shows a top view of an example of an adhesive bandage 24 that is an object of packaging and dispensing in the systems of the invention, along with a view of the dispensing tape 27 for comparison. The bandage 24 is shown attached to its protective tab 29. The dispensing tape 27 is of the same width or slightly larger than the bandage 24 and may be made of any material that can be rolled around the dispensing cylinder 18 and to which bandages may be attached with an adhesive. For example, the dispensing tape may be made of thin paper, of various plastic films such as a polypropylene, polyester, or polyvinyl, or of foil. The adhesive used to attach the bandage may be any conventional adhesive, so long as the bonding force is well below the tensile strength of the materials used in construction of the dispensing tape and adhesive bandage, so that the bandage can be easily removed from the dispensing tape without tearing either materials. The protective tab 29 may be limited to covering a portion of the bottom, adhesive portion of the bandage, and may be made of the same material as the dispensing tape and use the same adhesive to hold it to the bandage 24 that is used to hold the bandage 24 to the dispensing tape 27. To keep the pad in a sterile condition, the protective tab may also cover one adhesive end of the bandage completely, as well as the sterile pad and adhesive portions on either side of the pad, as well as a portion of the other adhesive end of the bandage.

FIG. 3B shows a bottom view of the exemplary bandage 24 with its protective tab 29. The bottom view also shows the sterile pad 26 centrally located in the bandage which is used to dress wounds, as well as the free adhesive portion 28 which is laterally located. To use the entire bandage 24, the protective tab 29 is removed in a similar manner to removing the bandage 24 from the dispensing tape 27, by grasping the protective tab 29 with the thumb and index finger and removing it, thereby exposing the other adhesive portion of the bandage. The second adhesive portion of the bandage can then be applied to the skin with one hand and with the bandage in place the protective tab can be removed with the same hand to completely expose the first adhesive portion of the bandage and the sterile pad, then the second adhesive portion can be applied to the skin to cover the wound. In other embodiments, the sterile pad 24 may be covered with a protective material lightly fixed with an adhesive to the dispensing tape to deter and prevent exposure to contamination.

FIGS. 4A-4C show another example of an adhesive bandage 24 with a grasping tab 31 that covers the majority of the bandage including the entire adhesive portion of one side of the bandage along with the sterile pad 26, with FIG. 4A representing a top view, FIG. 4B representing a bottom view, and FIG. 4C representing a side view. The embodiment shown in FIGS. 4A-4C can be used in a variety of dispensing means according to the invention, wherein the bandages are arranged in a continuous series. The grasping tab 31 provides a convenient handle for removing each bandage in the series, while shielding the sterile pad 26 from contamination, thus obviating the need for a separate strip of protective material. The protective tab is secured in place by being secured to an adhesive portion of the bandage on all sides of the pad.

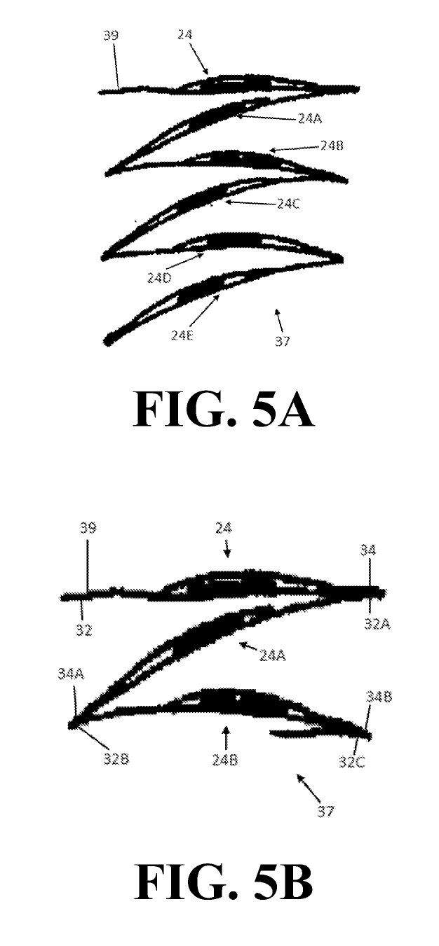

In another embodiment, the invention provides a system which packages bandages that are arranged in a continuous series through a stacked configuration to facilitate ease of dispensing. FIG. 5A shows a side view of a stack 37 of six bandages (24, 24A, 24B, 24C, 24D, 24E) arranged in a configuration according to the invention. Bandage 24 is the first bandage in the series, and the lead end in the series is end 39. FIG. 5B shows a close-up side view of the first three bandages in the stacked series according to the invention. The first bandage 24 in the stack 37 has a proximal end 32 and a distal end 34 according to the series, wherein the proximal end 32 of the first bandage 24 represents the leading end 39 of the stack. The distal end 34 of the first bandage 24 is attached to the next bandage 24A in the stack 37 by adhesive. In embodiments, the adhesive is disposed on the lower surface only of end 34 and adheres bandage 24 to the upper surface of bandage 24A at its proximal end 32A. The distal end 34A of the next bandage 24A is then attached to the proximal end 32B of the next bandage 24B (by adhesive disposed on the lower surface of 34A), which is attached at its distal end 34B to the next bandage's proximal end 32C (by adhesive disposed on the lower surface of 34B), and so on in the series. As a result, the bandages are arranged in a continuous series in an accordion-like fashion. The bandages may be attached together at their ends through the use of a light adhesive, or any adhesive used for bandages.

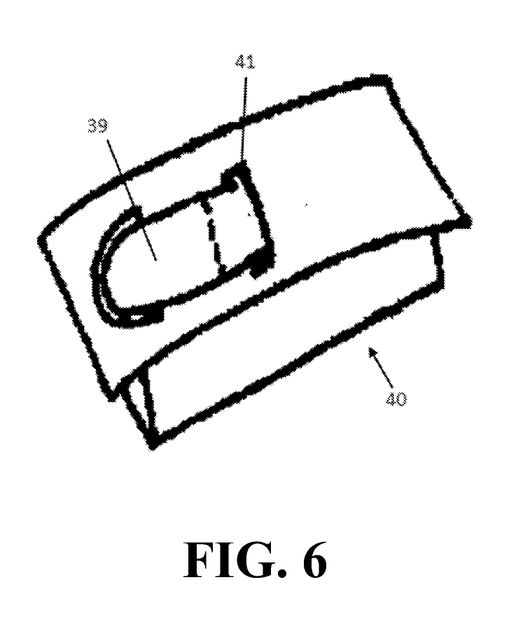

FIG. 6 shows a top, front, right side view of an embodiment of a dispensing means 40 for dispensing a stack 37 of bandages such as that shown in FIGS. 5A and 5B. The stack 37 is arranged in a dispensing means 40 with a box-like configuration that is approximately the same dimensions as the width and length of the stack of bandages. The leading end 39 of the stack 37 is advanced outside the dispensing means through a slit 41 at the top of the dispensing means 40. By grasping and pulling quickly the grasping tab 31 of the bandage 24 at the leading end 39 of the stack, the first bandage 24 may be removed from the dispensing means 40 by removing it from the next bandage 24A to which it is stuck, while simultaneously advancing the leading edge 32A of the next bandage 24A in the series outside the dispensing means 40 through the slit 41, so that the leading edge 32A of the next bandage 24A in the series becomes the leading end 39 of the stack, so that the next bandage may be similarly removed from the dispensing means 40. Thus, the retrieval of each bandage is dependent on the retrieval of the previous bandage in the stack 37. As one bandage is removed, the next bandage emerges from the slit in the dispenser, although facing the opposite side of the dispenser.

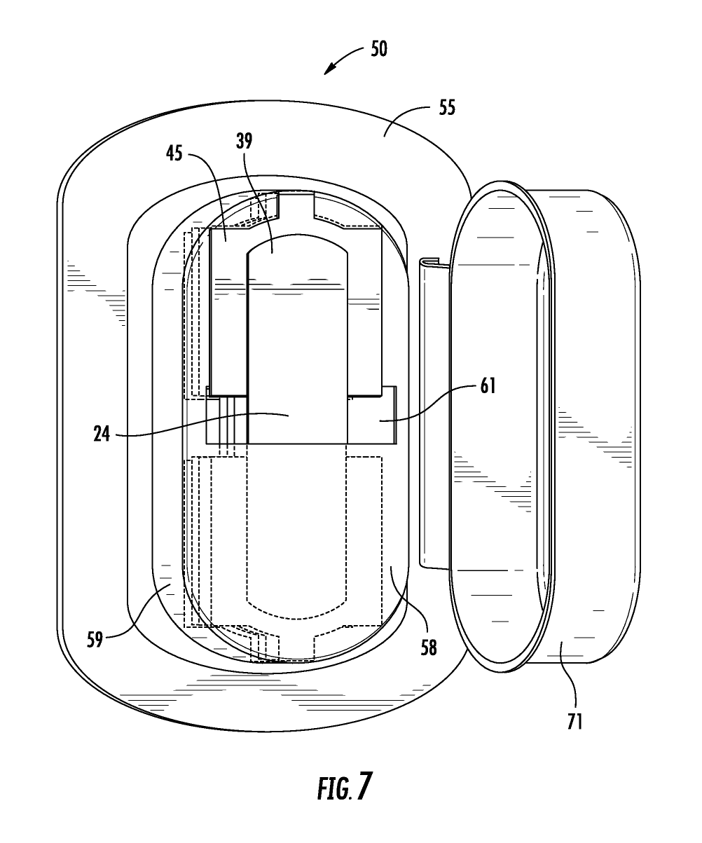

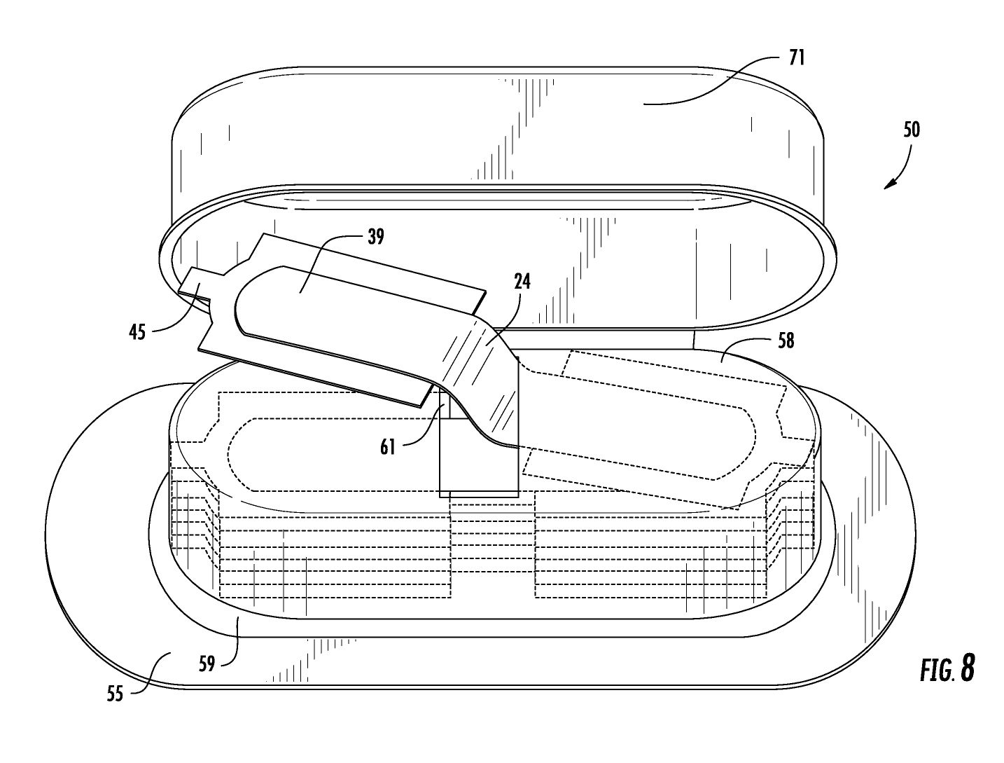

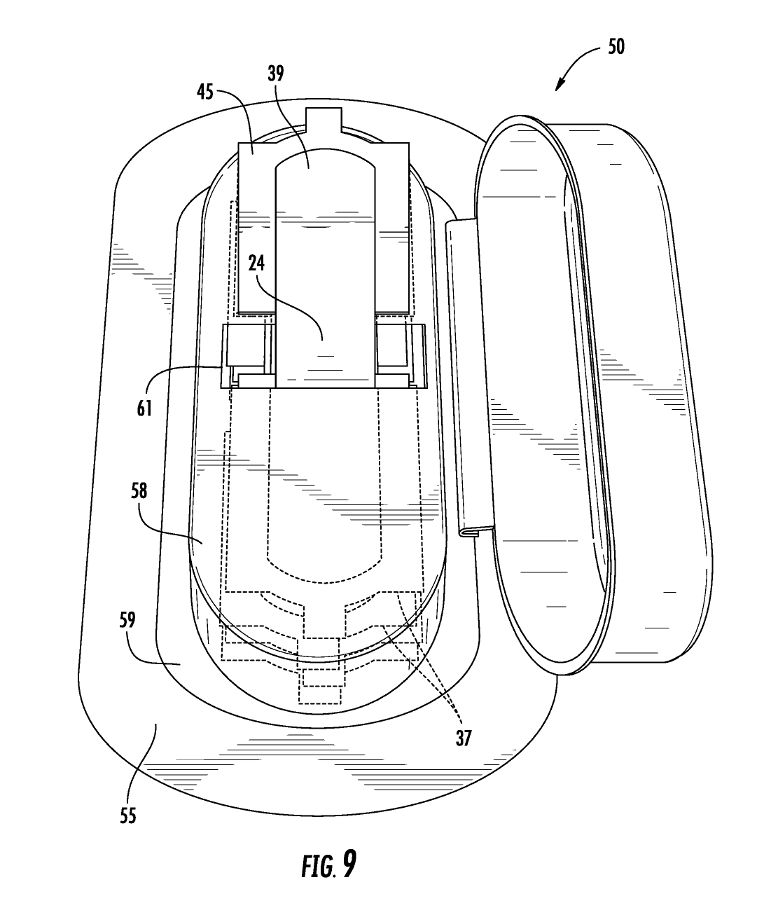

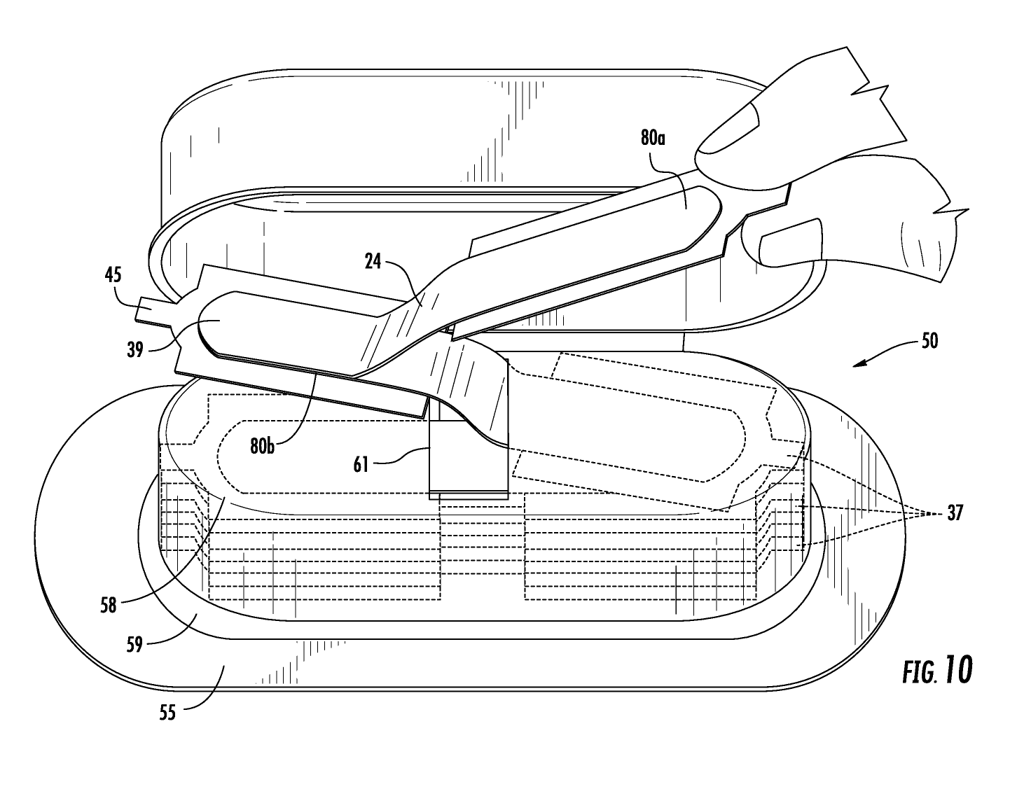

FIGS. 7-10 show another embodiment of a dispensing means for dispensing bandages in a stacked configuration such as that shown in FIGS. 5A and 5B, with FIG. 7 providing a top view, FIG. 8 providing a top, front view, FIG. 9 providing a left side view, and FIG. 10 providing a right side view.

The dispensing means 50 of FIGS. 7-10 optionally comprises a support substrate 55, generally providing a flat planar surface on which the dispenser can be disposed, that may be cardboard or other similar rigid piece of material. In embodiments, the support substrate can comprise means for attaching it to another surface, such as a wall, table, door, cabinet, counter top, or on a hanger for displaying the dispenser in a store. The means for attaching the support to a substrate can include a hole for accommodating a screw or an hanger, as well as double sided tape or even a hook and loop type fastening mechanism to name a few. The top surface of the flat piece 55 has an elongated dome 58 disposed on it that is of similar dimensions to the stack of bandages. In this respect, the support substrate acts as a bottom for the dispenser. In embodiments, the dome 58 comprises a flat portion 59 surrounding the circumference of the bottom of the dome for attachment of the dome 58 to the flat piece 55. Alternatively, the dome 58 can be a stand-alone device with a bottom instead of being attached to cardboard, in which case flat portion 59 is optional. The dome 58 may be made of plastic, and may be attached to the flat piece 55 at its flat portion 59 through the use of an adhesive. The dome 58 comprises a hole 61 through which the leading portion 39 of the stack of bandages may be advanced so that the leading portion 39 protrudes outside the dome 58, allowing for dispensing of bandages from the dispensing means 50 one at a time. The first bandage may be removed by grasping the leading end 39 of the stack at its grasping tab 45 between the thumb and index finger. Further, the side views of FIGS. 9 and 10 show the stack 37 of bandages inside the dome 58 of the dispensing means 50. An outer cover 71 (otherwise referred to as a housing), substantially the same shape as dome 58 can be incorporated into the dispensing means. In embodiments, the outer cover can be attached by a hinge or hingedly connected with the dome itself (FIG. 8), or the flat portion 59 around the base of the dome (FIG. 9), or the planar support member (FIG. 7).

Preferred embodiments comprise a system for dispensing adhesive bandages, as shown in FIG. 10, comprising: (a) a hollow dispenser 58 comprising an opening 61; (b) a series of adhesive bandages disposed in a stack 37 within the hollow dispenser 58; wherein each of the bandages 24 comprises a strip of material with a top and bottom surface and adhesive disposed on the bottom surface; wherein the adhesive releasably adheres a first end 39a of each bandage to the top surface of a second end 80b of another bandage in the stack, and wherein the bottom surface of the second end of each bandage is not adhered to another bandage in the stack (but to a protective strip 45); wherein, during use of the system 50, the second end 80a of a top bandage in the stack protrudes through the opening 61 of the dispenser 58 and when pulled by a user the top bandage 24 is removed from the stack and the second end 80b of another bandage in the stack is advanced to protrude through the opening 61 of the dispenser 58.

Any embodiment of the invention using a series of overlapping or partially overlapping bandages provided in a stack or a roll can comprise protective tabs on one or more of the bandages. The protective tabs can bear instructions for the user of the device, such as “pull here,” and/or can be numbered such that the bandages are consecutively numbered from highest to lowest to serve as an indicator to the user how many bandages remain to be dispensed.

Other embodiments of the dispensing means for dispensing the stacked configuration of bandages ranging from portable, semi-portable, to fixed fall within the scope of the present invention, and may be readily ascertained by the skilled artisan. While the embodiment depicted in FIGS. 7-10 is primarily intended for the consumer, other embodiments that are primarily intended for medical personnel, wherein the dispensing means is designed to hold a larger number of bandages and remain in a clinic also fall within the scope of the invention. Such embodiments may be made through modifications that are known in the art. Further, in another embodiment, the dispensing means may be mounted on a wall such as in the bathroom of the home or upside-down such as in underneath a cabinet.

The systems of the invention are used to package and dispense conventional adhesive bandages that are commonly known in the art. BAND-AID brand by JOHNSON & JOHNSON is most commonly known, but adhesive bandages by other manufacturers such as CURAD and 3M, as well as brands produced by popular drug store chains are also known. Materials used to manufacture conventional adhesive bandages as well as their various sizes, dimensions, and construction are common knowledge in the art, and therefore it is not necessary to recite them here. It is the packaging and dispensing of conventional adhesive bandages that this invention relates to. As the present invention may be used to package a wide array of numbers and sizes of bandages that are currently available, it should be reemphasized that the particular dimensions and numbers of adhesive bandages shown in the figures is merely exemplary. In some embodiments, the bandages that are used in the present invention may be shorter or longer in length relative to width and vice versa than those depicted herein. If desired, the systems of the invention can be configured to dispense multiple sized bandages.

The systems of the invention allow an individual with a wound or medical professional wishing to treat such an individual to quickly and intuitively remove a bandage from its container with a one hand, wherein the bandage is in a state ready for immediate application. The systems of the invention protect the bandages from contamination, thus obviating the need to package individual bandages within a separate envelope. The components of the systems are of simple construction and can easily be reproduced for manufacture and made available for the packaging and dispensing of conventional adhesive bandages.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention. Further, it is noted that all references cited in this disclosure, including any patents or published applications, are each hereby incorporated by reference herein in their entirety.

1. A system for dispensing adhesive bandages, comprising:
(a) a dispensing tape disposed as a roll;
(b) a series of partially overlapping adhesive bandages fixed to the dispensing tape, wherein each of the adhesive bandages has a first end releasably adhered to the dispensing tape and a second end that is not adhered to the dispensing tape; and
(c) a dispenser for housing the dispensing tape and the adhesive bandages.

2. The system of claim 1, wherein no overwrap encloses any adhesive bandage in its entirety.

3. The system of claim 1, wherein each adhesive bandage comprises a sterile pad and has a protective cover disposed over the sterile pad, which cover is releasably adhered to the adhesive bandage at only part of a bottom surface of the first end, at the second end on a bottom surface in its entirety, and along a perimeter in its entirety of a bottom surface of the bandage surrounding the sterile pad.

4. The system of claim 3, wherein the protective cover has a larger perimeter than a perimeter of the adhesive bandage to which it is adhered.

5. The system of claim 4, wherein the protective cover has one or more tabs for gripping the protective cover for removal from the adhesive bandage.

6. The system of claim 1, wherein the dispensing tape has a planar surface and a width equal to or larger than each of the adhesive bandages.

7. The system of claim 1, wherein the dispensing tape has a leading end and an opposite end and each of the adhesive bandages is disposed lengthwise on the dispensing tape with its first end oriented toward the leading end and its second end oriented toward the opposite end of the dispensing tape.

8. The system of claim 1, wherein the dispenser comprises a cylinder and the roll of dispensing tape is wrapped around the cylinder.

9. The system of claim 8, wherein the cylinder has two ends and further comprises two disks disposed parallel to one another, one at each end of the cylinder, and the disks have a larger circumference than the ends of the cylinder.

10. The system of claim 9 further comprising a cylindrical housing operably configured for engaging the disks at their circumference and for retaining in its entirety the roll of dispensing tape disposed on the cylinder.

11. The system of claim 1, wherein the dispensing tape is paper or plastic.

12. A system for dispensing adhesive bandages, comprising:
(a) a hollow dispenser with an opening;
(b) a series of adhesive bandages disposed in a stack within the dispenser;
wherein each bandage comprises a strip of material with a top and bottom surface and adhesive disposed on the bottom surface;
wherein the adhesive releasably adheres a first end of each bandage to the top surface of a second end of another bandage in the stack, and wherein the bottom surface of the second end of each bandage is not adhered to another bandage in the stack;
wherein, during use of the system, the second end of a top bandage in the stack protrudes through the opening of the dispenser and when pulled by a user the top bandage is removed from the stack and the second end of another bandage in the stack is advanced to protrude through the opening of the dispenser.

13. The system of claim 12, wherein no overwrap encloses any adhesive bandage in its entirety.

14. The system of claim 12, wherein each adhesive bandage comprises a sterile pad and has a protective cover disposed over the sterile pad, which cover is releasably adhered to the adhesive bandage at only part of the bottom surface of the first end, at the bottom surface of the second end in its entirety, and along the bottom surface in its entirety which surrounds the sterile pad.

15. The system of claim 14, wherein the protective cover has a larger perimeter than a perimeter of the adhesive bandage to which it is adhered.

16. The system of claim 15, wherein the protective cover has one or more tabs for gripping the protective cover for removal from the adhesive bandage.

17. The system of claim 12, wherein the dispenser is mounted to a planar member.

18. The system of claim 17, wherein the dispenser further comprises an outer cover attached by a hinge to the planar member.

19. The system of claim 12 further comprising a dispenser outer cover.

20. The system of claim 18, wherein the dispenser outer cover is attached by a hinge to the dispenser.
